(19) United States
(12) Patent Application Publication
(10) Pub. No.: US 2021/0388450 A1
(43) Pub. Date: Dec. 16, 2021

(54) BIOMARKER PANEL FOR DETERMINING MOLECULAR SUBTYPE OF LUNG CANCER, AND USE THEREOF

(71) Applicant: SAMSUNG LIFE PUBLIC WELFARE FOUNDATION, Seoul (KR)

(72) Inventors: Myung Ju AHN, Seoul (KR); Hae Ock LEE, Seoul (KR); Na Young KIM, Seoul (KR)

(73) Assignee: SAMSUNG LIFE PUBLIC WELFARE FOUNDATION, Seoul (KR)

(21) Appl. No.: 17/289,490

(22) PCT Filed: Oct. 25, 2019
(86) PCT No.: PCT/KR2019/014168

§ 371 Date: Apr. 28, 2021

(30) Foreign Application Priority Data
Oct. 29, 2018 (KR) 10-2018-0130245

(51) Int. Cl. C12Q 1/6886

(52) U.S. Cl. CPC C12Q 1/6886; C12Q 2600/158; C12Q 2600/118; C12Q 2600/112

(57) ABSTRACT

The present invention relates to a biomarker panel for determining a molecular subtype of cancer and a method of predicting prognosis of cancer using the panel. The biomarker panel identifies molecular subtypes of tumors by selecting and analyzing only information on tumor cells from single-cell transcriptome data derived from patients with early lung cancer, thereby allowing prediction of the prognosis of lung cancer and prediction of the response to anti-cancer agents. Therefore, the biomarker panel may be used for selecting a treatment regimen.

### TECHNICAL FIELD

The present disclosure relates to a biomarker panel for determining molecular subtypes of lung cancer and a method of predicting prognosis of cancer using the panel.

### BACKGROUND ART

Lung cancer is one of the most common malignancies worldwide. According to statistics compiled in the United States in 2017, lung cancer has the highest cancer mortality rate in both males and females, and the incidence rate is also the second highest in both males and females. There are two types of lung cancer: small cell and non-small cell. Non-small cell lung cancer accounts for approximately 83% of all lung cancer cases, and has a 5-year survival rate of 21%, which makes it a cancer with the worst prognosis among solid cancers. More than 40% of non-small cell lung cancers are found in the metastatic stage (stage IV) at diagnosis, and thus only a few patients may be treated surgically. When surgery is not possible, the 5-year survival rate is very low at only a range of 15.7% to 17.4%, even with chemotherapy, and thus existing surgeries, radiation therapies, anticancer chemotherapies, and combination therapies thereof have limitations in treatment. As the understanding of the pathogenesis of lung cancer has broadened, non-small cell lung cancers can be classified into several subtypes depending on the presence or absence of mutation in genes such as epidermal growth factor receptor (EGFR), anaplastic lymphoma kinase (ALK), ROS1 or PD-1, and targeted therapies using these have appeared, which has led to significant changes in lung cancer treatment.

One of the reasons why heterogeneity in response to treatment is important is because treatment is attempted in a way that is applicable to all. In this regard, not many studies have been conducted on the basic molecular mechanisms that lead to differences in cancer severity and treatment outcomes. Therefore, there is a need to differentiate the molecular subtypes of lung cancer and reveal the relationship between genetic modifications, survival outcomes, and postoperative recurrence patterns according to subtypes, to develop a treatment plan according to genetic alteration and use the plan for customized treatment.

### DESCRIPTION OF EMBODIMENTS

### Technical Problem

Provided is a biomarker panel including an agent measuring the level of at least two biomarkers selected from the group consisting of S100A4, TMSB10, KRT19, RAC1, S100A2, MDK, ISG15, KRT7, CLDN3, CDKN2A, and IFI27.

Provided is a method of predicting prognosis of cancer, the method including measuring the level of at least two biomarkers selected from the group consisting of S100A4, TMSB10, KRT19, RAC1, S100A2, MDK, ISG15, KRT7, CLDN3, CDKN2A, and IFI27 from a sample isolated from an individual; and comparing the level of the biomarkers with a corresponding results of the corresponding markers in a control sample.

Provided is a method of determining a molecular subtype of cancer, the method including obtaining single-cell transcriptome data from a sample isolated from an individual; and extracting a subset of genes from the data.

Provided is use of an agent for manufacturing a biomarker panel for predicting prognosis of cancer, wherein the agent measures the level of at least two biomarkers selected from the group consisting of S100A4, TMSB10, KRT19, RAC1, S100A2, MDK, ISG15, KRT7, CLDN3, CDKN2A, and IFI27.

### Solution to Problem

According to an aspect of the present disclosure, provided is a biomarker panel including an agent measuring the level of at least two biomarkers selected from the group consisting of S100A4, TMSB10, KRT19, RAC1, S100A2, MDK, ISG15, KRT7, CLDN3, CDKN2A, and IFI27. In one embodiment, the biomarker panel may further include at least one biomarker selected from the group consisting of AGR2, SOX4, C15orf48, CRIP2, HMGA1, TUBB, MARCKSL1, and IGFBP3. In some embodiments, the biomarker panel may further include at least one biomarker selected from the group consisting of CSTB, S100A16, COL1A1, SPATS2L, HN1, SPINT2, PTGS2, ANXA2, and TAGLN2.

As used herein, the term “biomarker panel” is constructed using any combination of biomarkers for the diagnosis of lung cancer, and the combination may refer to an entire set, or any subset or subcombination thereof. That is, a biomarker panel may refer to a set of biomarkers, and may refer to any form of the biomarker that is measured. Thus, when S100A4 is part of a biomarker panel, either S100A4 mRNA or S100A4 protein, for example, may be considered to be part of the panel. While individual biomarkers are useful as diagnostics, combination of biomarkers may sometimes provide greater value than a single biomarker alone in determining a particular status. Particularly, the detection of a plurality of biomarkers in a sample may increase the sensitivity and/or specificity of the test. Thus, in various embodiments, a biomarker panel may include 1, 2, 3, 4, 5, 6, 7, 8, 9, 10, 11, or more types of biomarkers. In some embodiments, the biomarker panel consists of a minimum number of biomarkers to generate a maximum amount of information. Accordingly, in various embodiments, the biomarker panel consists of 1, 2, 3, 4, 5, 6, 7, 8, 9, 10, 11, or more types of biomarkers. When a biomarker panel consists of “a set of biomarkers”, no biomarker other than those of the set is present. In one embodiment, the biomarker panel consists of 1 biomarker disclosed herein. In some embodiments, the biomarker panel consists of 2 biomarkers disclosed herein. In some embodiments, the biomarker panel consists of 3 biomarkers disclosed herein. In some embodiments, the biomarker panel consists of 4 or more biomarkers disclosed herein. The biomarkers of the present disclosure show a statistically significant difference in lung cancer diagnosis. In one embodiment, diagnostic tests that use these biomarkers alone or in combination show a sensitivity and specificity of at least about 85%, at least about 90%, at least about 95%, at least about 98%, and about 100%.

The biomarkers may be obtained from single-cell transcriptome data. Also, the biomarkers may be for diagnosing cancer, and the cancer may be lung cancer. In particular, the molecular subtypes of tumor cells may be classified according to the gene expression with a gene expression value of epithelial cells derived from normal tissues as a control from the results of analyzing gene expression data of single cells derived from tumor tissues of early lung cancer patients.

As used herein, the term “molecular subtype” refers to subtypes of a tumor that are characterized by distinct molecular profiles, e.g., gene expression profiles. In one embodiment, the molecular subtype may be selected from 6,352 single cells derived from tumor tissues of early lung cancer patients. The molecular subtype may be a molecular subtype of tumor cell classified according to the gene expression by setting a gene expression value of epithelial cells derived from normal tissues as a control. Here, the molecular subtype may be classified into state 1, state 2, and state 3 depending on the functional properties. Tumor cells corresponding to the state 1 and state 3 maintain functional properties of normal epithelial cells, whereas tumor cells corresponding to the state 2 exhibit functions related to cancer metastasis and development, such as cell migration, apoptosis, and cell proliferation.

The agent measuring the level of a biomarker may be a primer pair, a probe, or an antisense nucleotide. In particular, the agent may be an agent for measuring an mRNA level of the biomarker gene and may be a primer pair, a probe, or an antisense nucleotide that specifically binds to the gene. In one embodiment, the biomarker panel may include at least 11 types of primer pairs, probes, or antisense nucleotides, and each of the primer pairs, probes, or antisense nucleotides may specifically bind to S100A4, TMSB10, KRT19, RAC1, S100A2, MDK, ISG15, KRT7, CLDN3, CDKN2A, and IFI27.

The agent measuring the level of a biomarker may be an antibody. The antibody may be a monoclonal antibody and, for example, may be a monoclonal antibody capable of specifically binding to any of the biomarkers. In one embodiment, the biomarker panel may include at least 11 types of antibodies, and each of the antibodies is capable of specifically binding to S100A4, TMSB10, KRT19, RAC1, S100A2, MDK, ISG15, KRT7, CLDN3, CDKN2A, and IFI27.

According to another aspect of the present disclosure, provided is a method of predicting prognosis of cancer, the method including measuring the level of at least two biomarkers selected from the group consisting of S100A4, TMSB10, KRT19, RAC1, S100A2, MDK, ISG15, KRT7, CLDN3, CDKN2A, and IFI27 from a sample isolated from an individual; and comparing the level of the biomarkers with a corresponding results of the corresponding markers in a control sample. Details of the biomarker are the same as described above.

The cancer may be lung cancer, for example, early lung cancer. Also, the lung cancer may be, for example, adenocarcinoma, squamous cell carcinoma, large cell carcinoma, adenosquamous cell carcinoma, sarcoma cancer, carcinoid tumor, salivary gland cancer, unclassified cancer, or small cell lung cancer.

The method according to an embodiment includes measuring the level of at least two biomarkers selected from the group consisting of S100A4, TMSB10, KRT19, RAC1, S100A2, MDK, ISG15, KRT7, CLDN3, CDKN2A, and IFI27 from a sample isolated from an individual. In some embodiments, the method may further include measuring the level of at least two biomarkers selected from the group consisting of AGR2, SOX4, C15orf48, CRIP2, HMGA1, TUBB, MARCKSL1, IGFBP3, CSTB, S100A16, COL1A1, SPATS2L, HN1, SPINT2, PTGS2, ANXA2, and TAGLN2.

The individual is a subject for diagnosis of cancer and may refer to, for example, a subject for predicting the likelihood of cancer, a subject for diagnosing the condition of cancer, a subject for determining prognosis prediction, a subject for determining an administration dose of a drug for preventing or treating cancer, or a subject for determining a treatment method according to the progress of cancer. The individual may be a vertebrate animal, for example, a mammal, an amphibian, a reptile, or a bird, and more specifically, may be a mammal, for example, a human (Homo sapiens). The sample may include a sample such as tissue, cells, whole blood, serum, plasma, saliva, sputum, cerebrospinal fluid, or urine separated from the individual.

The measuring of the level of the biomarkers may be performed by measuring an mRNA level or a protein level of at least two biomarker genes selected from the group consisting of S100A4, TMSB10, KRT19, RAC1, S100A2, MDK, ISG15, KRT7, CLDN3, CDKN2A, IFI27, AGR2, SOX4, C15orf48, CRIP2, HMGA1, TUBB, MARCKSL1, IGFBP3, CSTB, S100A16, COL1A1, SPATS2L, HN1, SPINT2, PTGS2, ANXA2, and TAGLN2. Particularly, the measuring of an mRNA level is a process of verifying the presence and expression levels of mRNA of genes in a sample of an individual to diagnose cancer, and measures the amount of mRNA. The analysis methods for the above purpose may include reverse transcription polymerase chain reaction (RT-PCR), competitive RT-PCR, real-time RT-PCR, RNase protection assay (RPA), Northern blotting, or DNA chip, reverse transcription polymerase chain reaction (RT-PCR), competitive RT-PCR, real-time RT-PCR, RNase protection assay (RPA), Northern blotting, or DNA chip. Also, the measuring of a protein level is a process of verifying the presence and expression level of a marker protein for diagnosing cancer in a sample of an individual to diagnose cancer. The amount of protein may be confirmed by using an antibody that specifically binds to the marker protein, and the protein expression level itself may be measured without using the antibody. The protein level measurement or comparative analysis methods may include protein chip analysis, immunoassay, ligand binding assay, Matrix Desorption/Ionization Time of Flight Mass Spectrometry (MALDI-TOF) analysis, Surface Enhanced Laser Desorption/Ionization Time of Flight Mass Spectrometry (SELDI-TOF), radioimmunoassay, radioimmunodiffusion, Ouchterlony immunodiffusion, rocket immunoelectrophoresis, immunohistochemical staining, complement fixation assay, two-dimensional electrophoresis, liquid chromatography-Mass Spectrometry (LC-MS), liquid chromatography-Mass Spectrometry/Mass spectrometry (LC-MS/MS), Western blotting, and enzyme linked immunosorbent assay (ELISA).

The method according to an embodiment includes the comparing the level of the biomarkers with a corresponding result of the corresponding markers in a control sample. For example, when the biomarker is overexpressed as compared with a control sample, the prognosis of the cancer may be determined as poor. In one embodiment, it was confirmed that there is a relationship between the expression of genes corresponding to state 2 among the molecular subtypes of lung cancer and the decrease in the survival rate of the patient. Particularly, it was confirmed that the association is maximized when the patient is an early lung cancer patient. Therefore, the expression level of the biomarker may be used as an index to predict the prognosis of the patient.

According to another aspect of the present disclosure, provided is a method of determining a molecular subtype of cancer, the method including obtaining single-cell transcriptome data from a sample isolated from an individual; and extracting a subset gene from the data. The cancer may be lung cancer.

The method according to an embodiment includes obtaining single-cell transcriptome data from a sample isolated from an individual. A conventional method of determining a molecular subtype using bulk tissue transcriptome data has a problem of difficulty in reflecting the characteristics of tumor itself. Thus, in one embodiment, gene expression data produced from single cells derived from early lung cancer patients and single cells derived from normal lung tissue adjacent to tumor were used. Also, for analysis of pure tumor cells, analysis was performed using the gene expression data for tumor cells derived from tumor tissue and epithelial cells derived from normal lung tissue. That is, the molecular subtype of lung cancer was determined by extracting only information on pure tumor cells from single-cell transcriptome data derived from lung cancer patients, specifically, early lung cancer patients, and thus the method according to an embodiment allows more accurate analysis than determining molecular subtypes using the conventional bulk tissue transcriptome data.

The method according to an embodiment includes extracting a subset of genes from the data. The method according to an embodiment may further include selecting a signature gene from the extracted subset genes. In particular, the extracting a subset of genes may be performed by determining state difference in tumor cells based on normal cells. For example, when the expression in tumor cells is increased as compared with that of normal cells, it can be extracted as a subset of genes. Here, the subset genes extracted by the above method may exhibit an expression level specific to tumor cells, and may exhibit characteristics associated with cancer metastasis or development.

As used herein, the term “signature” refers to a sign of a biomarker for a given diagnostic test, which contains a series of markers, each marker having different levels in the populations of interest. The different levels may refer to different means of the marker levels for individuals in two or more groups, or different changes in two or more groups, or a combination of both. Here, the signature gene is a gene selected to classify molecular subtypes between tumor cells, and may have characteristics of tumor cell states. For example, a set of genes that are each overexpressed in state 1, state 2, and state 3 of a tumor cells may be named as a signature gene according to the state. Therefore, the set of genes forming the biomarker panel according to an embodiment may be a signature gene of state 2.

According to another aspect of the present disclosure, provided is use of an agent for manufacturing a biomarker panel for predicting prognosis of cancer, wherein the agent measures the level of at least two biomarkers selected from the group consisting of S100A4, TMSB10, KRT19, RAC1, S100A2, MDK, ISG15, KRT7, CLDN3, CDKN2A, and IFI27. Details of the cancer, biomarker panel, biomarker, and agent measuring the level of the biomarkers are the same as described above. In one embodiment, the biomarker may further include at least one biomarker selected from the group consisting of AGR2, SOX4, C15orf48, CRIP2, HMGA1, TUBB, MARCKSL1, and IGFBP3. In some embodiments, the biomarker may further include at least one biomarker selected from the group consisting of CSTB, S100A16, COL1A1, SPATS2L, HN1, SPINT2, PTGS2, ANXA2, and TAGLN2.

### Advantageous Effects of Disclosure

a biomarker panel according to an embodiment identifies molecular subtypes of tumors by selecting and analyzing only information on tumor cells from single-cell transcriptome data derived from patients with early lung cancer, thereby allowing prediction of the prognosis of lung cancer and prediction of the response to anti-cancer agents. Therefore, the biomarker panel may be used for selecting a treatment regimen.

### BRIEF DESCRIPTION OF DRAWINGS

FIG. 1 shows diversity of cell types in tumor tissue (tLung) and normal tissue (nLung) in patients with early lung cancer;

FIG. 2A shows graphs for determining cell subtypes of tumor cells and normal epithelial cells using single-cell transcriptome data of patients with early lung cancer, and FIG. 2B shows graphs for differentiating molecular subtypes of tumor cells based on gene expression by extracting only epithelial cells;

FIG. 3A shows the results of selecting molecular subtype-specific marker genes of tumor cells, showing an expression pattern of marker genes corresponding to the top 15 gene expressions for each molecular subtype, and FIG. 3B shows graphs showing functional characteristics of molecular subtype-specific marker genes for each molecular subtype;

FIG. 4A shows graphs of survival curves of patients with lung adenocarcinoma, and FIG. 4B shows graphs of survival curves of patients with lung squamous cell carcinoma.

FIG. 5 shows the results of measuring expression levels of single cells and protein levels of selected marker genes specific to tumor cell state 2 (tS2) epithelial subtype with respect to tissue samples of patients with lung adenocarcinoma (LUAD).

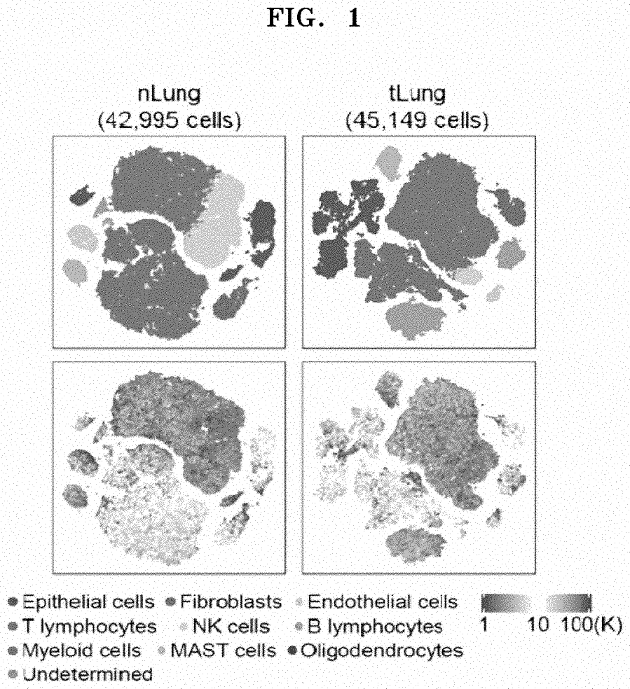

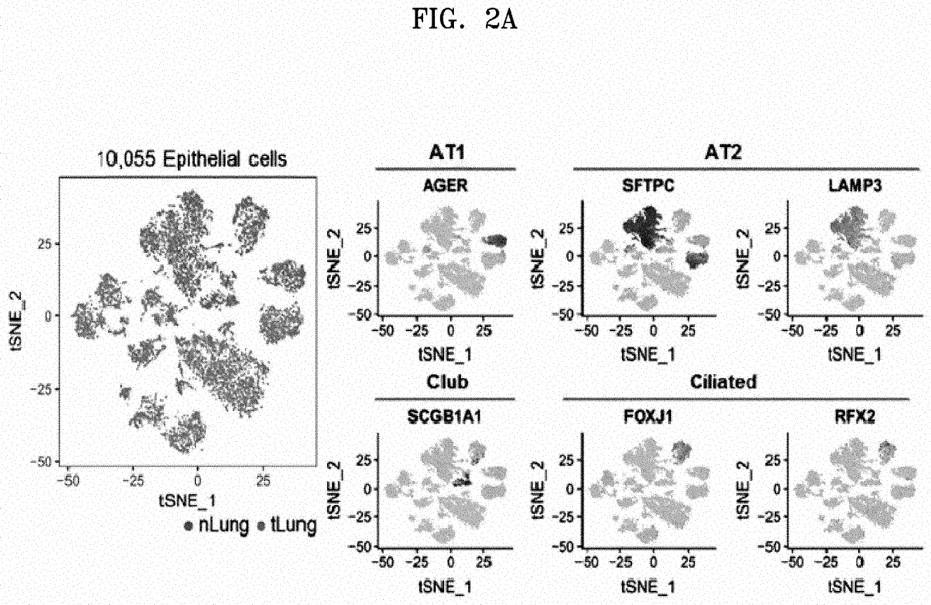

### MODE OF DISCLOSURE

Hereinafter, the present disclosure will be described in more detail with reference to examples. The examples are for only descriptive purposes, and it will be understood by those skilled in the art that the scope of the present disclosure is not construed as being limited to the examples.

### EXAMPLE

### Example 1. Determination of Major Subtypes of Lung Adenocarcinoma Through Single-Cell Transcriptome Analysis

1-1. Sample Preparation

The following experiments were reviewed and approved by the Institutional Review Board (IRB) of Samsung Medical center (IRB no. 2010-04-039-041) and performed on 12 patients pathologically diagnosed with lung adenocarcinoma. In particular, tumors and normal tissues were obtained from patients diagnosed as to undergo lung adenocarcinoma conserving surgery at Samsung Medical Center (in Seoul, Korea) from the patients that had not received prior treatment. On the day of surgery, 11 primary tumor samples and 11 adjacent normal lung tissues (10 pairs, non-paired tumor tissue, non-paired normal lung tissue) were collected using mechanical dissociation and enzymatic digestion to obtain a single cell suspension. Then, dead cells were removed by cell isolation using Ficoll-Paque PLUS (GE Healthcare, Sweden).

1-2. Single Cell RNA Sequencing and Pre-Treatment

According to the experimental protocol provided by the manufacturer, 3′ single cell RNA sequencing was performed using the GemCode system (10× genomics, Pleasanton, Calif., USA) targeting a total of 5,000 cells from each cell suspension. GemCode single cell RNA sequencing reads were mapped on to the GRCh38 human reference genome using the Cell Ranger toolkit (version 2.1.0). Three quality measures were applied: mitochondrial gene (less than 20%), unique molecular identifiers (UMI), and gene count (ranging from 1000 to 150,000 and 200 to 10,000) calculated from the gene-cell-barcode matrix that did not undergo standardization. The UMI count for the genes in each cell was log-normalized to transcript per million (TPM)-like values, and then the gene expression was quantified in the scale of log 2(TPM+1) as described in Haber et al., (2017).

1-3. Unsupervised Clustering

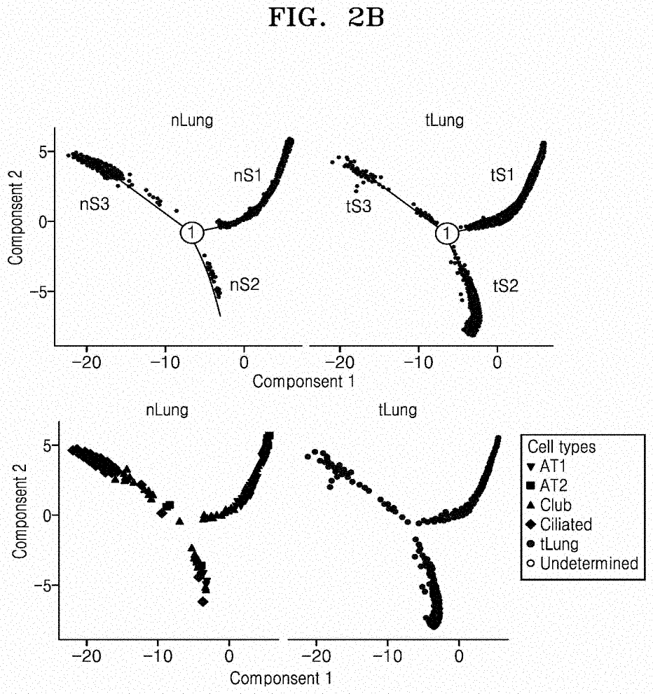

The results of the single cell RNA-sequencing in Examples 1-2 were analyzed using unsupervised clustering, and as a result, subclusters were shown as largely divided by tissue origin, tumor, and normal region (FIG. 2A). Particularly, variable genes were selected from the R package Seurat R toolkit (Butler et al., 2018) (https://satijalab.org/seurat/) and used to compute principal components (PC). A subset of important principal components for cell-clustering were selected by R function JackStraw of the Seurat package and were used for t-distributed stochastic neighbor embedding (tSNE) visualization. The cell type of each cluster was defined with an expression level of known marker genes. As a result, normal epithelial cells consisted mainly of four clusters which express normal epithelial cell type makers known as AGER, SFTPC, LAMP3, SCGB1A1, FOXJ1, and RFX2. In comparison, epithelial tumor cells formed patient-specific clusters.

1-4. Inference of Tumor Cell State Using Trajectory Analysis

An unsupervised trajectory analysis was performed using Monocle (version 2) to infer the development trajectory of lung epithelial cells (FIG. 2B). In particular, subsets of EPCAM and cell clusters were extracted from single cell RNA sequencing data regarding tumor and normal lung tissue samples for intensive analysis on tumor cells. Variable genes selected by Seurat were applied to the Monocle (version 2) algorithm (Qiu et al., 2017) to determine the differential tumor cell states referenced against normal epithelial cells. The gene-cell matrix in the scale of UMI counts was loaded into Monocle by input, and then, an object was created with the parameter “expressionFamily=negbinomial.size” by applying the newCellDataSet function. The epithelial cell trajectory was inferred using default parameters of Monocle after dimension reduction and cell ordering.

As a result, in the case of normal epithelial cells, ciliated epithelial cells were located at the opposite end of alveolar cells, indicating a distinct differentiation program. Club cells derived from normal lungs were located in the middle, and indicated an intermediate differentiation state (Chen and Fine, 2016; Cheung and Nguyen, 2015). Parallel analysis of tumor epithelial cells reflects the branched structure of normal epithelial cells, and has separate tumor cells (state 2) located at opposite ends of the two branches (state 1 and state 3) that are mainly contained in the epithelial cells of normal lungs. Overall tumor cells of state 1 and 3 followed the normal differentiation programs, whereas the tumor cells of state 2 diverged from the normal transcriptional programs.

1-5. Definition of Signature for Molecular Subtypes

To identify genes specific to each tumor cell state, log₂ (fold change) (log₂FC) between two groups were calculated (cell state vs other state). Importance of the difference was determined by Student's t-distribution and t-test with Bonferroni correction. In order to classify molecular subtype between tumor cells, genes having a FDR value and P value less than 0.01 and log₂FC>1 were selected as signature genes. The selected genes were classified according to a functional gene set using DAVID (https://david.ncifcrf.gov/) pathway enrichment analysis.

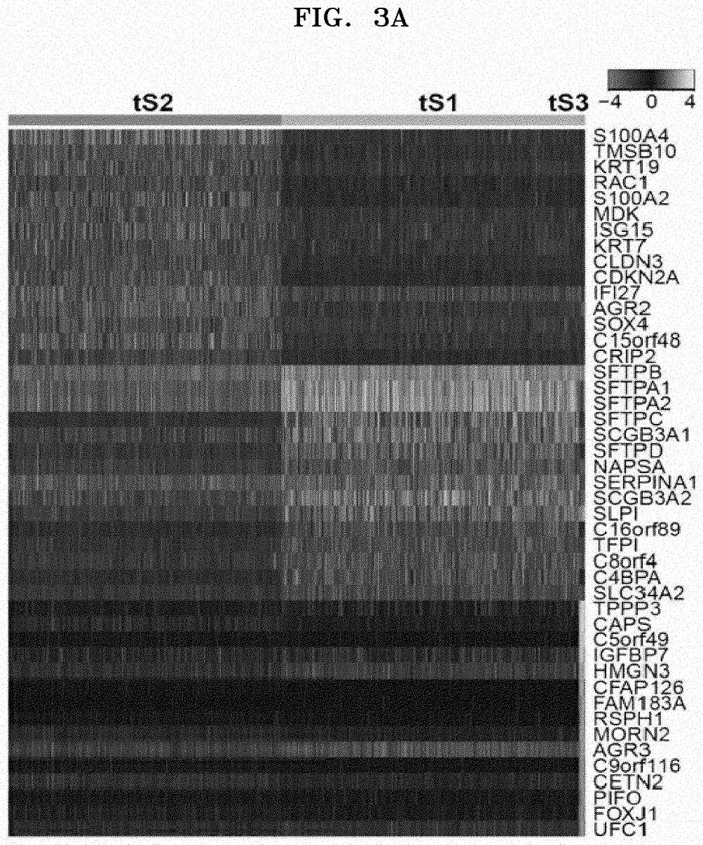

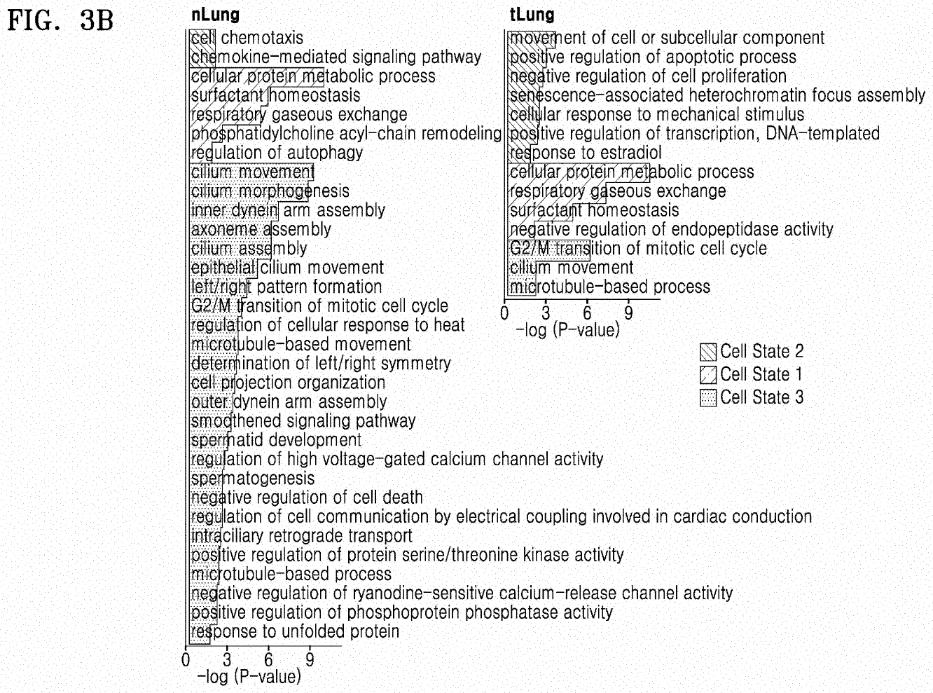

As a result, 19, 28, and 79 gene sets were identified as signature genes that significantly increase in each of tumor cell states 1, 2, and 3 (Tables 1 to 3). Most of genes associated with tumor cell sates 1 and 3 fall within the cell-specific functional categories of surfactant homeostasis, alveolar development of the lungs, and bacterial movement, whereas genes associated with tumor cell state 2 were abundantly present in the set of tumor-related gene of cell movement and cell death processes (FIG. 3B). In this regard, the tumor epithelial cells of lung adenocarcinoma (LUAD) may be classified into three main subtypes according to their unique characteristics indicating the differentiation pathway as well as the possibility of metastasis.

TABLE 1

| | Tumor cells (state 1) vs other state | | | Tumor cells (state 1) vs normal cells | | |
|---|---|---|---|---|---|---|
| Gene | log₂ FC | P-value | FDR | log₂ FC | P-value | FDR |
| SFTPB | 3.199 | 0 | 0 | 0.601 | 0 | 0 |
| SFTPA1 | 2.768 | 0 | 0 | −0.429 | 0 | 0 |
| SFTPA2 | 2.616 | 0 | 0 | −0.413 | 0 | 0.001 |
| SFTPC | 2.108 | 0 | 0 | −4.082 | 0 | 0 |
| SCGB3A1 | 1.779 | 0 | 0 | 0.842 | 0 | 0 |
| SFTPD | 1.665 | 0 | 0 | −0.5 | 0 | 0 |
| NAPSA | 1.61 | 0 | 0 | 0.641 | 0 | 0 |
| SERPINA1 | 1.569 | 0 | 0 | 1.322 | 0 | 0 |
| SCGB3A2 | 1.545 | 0 | 0 | 0.554 | 0 | 0 |
| SLPI | 1.48 | 0 | 0 | −1.234 | 0 | 0 |
| C16orf89 | 1.323 | 0 | 0 | 0.333 | 0 | 0 |
| TFPI | 1.3 | 0 | 0 | 1.15 | 0 | 0 |
| C8orf4 | 1.211 | 0 | 0 | 0.889 | 0 | 0 |
| C4BPA | 1.181 | 0 | 0 | 0.368 | 0 | 0 |
| SLC34A2 | 1.172 | 0 | 0 | 0.463 | 0 | 0 |
| PIGR | 1.165 | 0 | 0 | 0.062 | 0.125 | 1 |
| HOPX | 1.146 | 0 | 0 | 1.034 | 0 | 0 |
| SFTA1P | 1.086 | 0 | 0 | 0.325 | 0 | 0 |
| CTSH | 1.042 | 0 | 0 | −0.172 | 0 | 0.052 |

TABLE 2

| | Tumor cells (state 2) vs other state | | | Tumor cells (state 2) vs normal cells | | |
|---|---|---|---|---|---|---|
| Gene | log₂ FC | P-value | FDR | log₂ FC | P-value | FDR |
| S100A4 | 1.83 | 0 | 0 | 0.756 | 0 | 0 |
| TMSB10 | 1.829 | 0 | 0 | 2.293 | 0 | 0 |
| KRT19 | 1.611 | 0 | 0 | 1.899 | 0 | 0 |
| RAC1 | 1.451 | 0 | 0 | 1.769 | 0 | 0 |
| S100A2 | 1.429 | 0 | 0 | 1.659 | 0 | 0 |
| MDK | 1.403 | 0 | 0 | 2.514 | 0 | 0 |
| ISG15 | 1.399 | 0 | 0 | 2.005 | 0 | 0 |
| KRT7 | 1.398 | 0 | 0 | 1.867 | 0 | 0 |
| CLDN3 | 1.383 | 0 | 0 | 1.598 | 0 | 0 |
| CDKN2A | 1.339 | 0 | 0 | 1.674 | 0 | 0 |
| IFI27 | 1.337 | 0 | 0 | 2.833 | 0 | 0 |
| AGR2 | 1.291 | 0 | 0 | 0.871 | 0 | 0 |
| SOX4 | 1.284 | 0 | 0 | 2.091 | 0 | 0 |
| C15orf48 | 1.237 | 0 | 0 | 1.834 | 0 | 0 |
| CRIP2 | 1.193 | 0 | 0 | 1.123 | 0 | 0 |
| HMGA1 | 1.173 | 0 | 0 | 1.393 | 0 | 0 |
| TUBB | 1.152 | 0 | 0 | 1.393 | 0 | 0 |
| MARCKSL1 | 1.136 | 0 | 0 | 1.864 | 0 | 0 |
| IGFBP3 | 1.103 | 0 | 0 | 1.124 | 0 | 0 |
| CSTB | 1.099 | 0 | 0 | 1.336 | 0 | 0 |
| S100A16 | 1.093 | 0 | 0 | 1.634 | 0 | 0 |
| COL1A1 | 1.073 | 0 | 0 | 1.257 | 0 | 0 |
| SPATS2L | 1.065 | 0 | 0 | 1.056 | 0 | 0 |
| HN1 | 1.062 | 0 | 0 | 1.752 | 0 | 0 |
| SPINT2 | 1.05 | 0 | 0 | 0.928 | 0 | 0 |
| PTGS2 | 1.043 | 0 | 0 | 1.011 | 0 | 0 |
| ANXA2 | 1.024 | 0 | 0 | 0.749 | 0 | 0 |
| TAGLN2 | 1.007 | 0 | 0 | 1.277 | 0 | 0 |

TABLE 3

| | Tumor cells (state 3) vs other state | | | Tumor cells (state 3) vs normal cells | | |
|---|---|---|---|---|---|---|
| Gene | log₂ FC | P-value | FDR | log₂ FC | P-value | FDR |
| TPPP3 | 3.805 | 0 | 0 | 2.047 | 0 | 0 |
| CAPS | 3.431 | 0 | 0 | 2.599 | 0 | 0 |
| C5orf49 | 3.027 | 0 | 0 | 2.515 | 0 | 0 |
| IGFBP7 | 2.676 | 0 | 0 | 1.825 | 0 | 0 |
| HMGN3 | 2.327 | 0 | 0 | 1.918 | 0 | 0 |
| CFAP126 | 2.239 | 0 | 0 | 1.887 | 0 | 0 |
| FAM183A | 2.096 | 0 | 0 | 1.431 | 0 | 0 |
| RSPH1 | 2.085 | 0 | 0 | 1.494 | 0 | 0 |
| MORN2 | 2.082 | 0 | 0 | 1.696 | 0 | 0 |
| AGR3 | 2.076 | 0 | 0 | 1.781 | 0 | 0 |
| C9orf116 | 1.955 | 0 | 0 | 1.499 | 0 | 0 |
| CETN2 | 1.955 | 0 | 0 | 1.59 | 0 | 0 |
| PIFO | 1.863 | 0 | 0 | 1.34 | 0 | 0 |
| FOXJ1 | 1.852 | 0 | 0 | 1.601 | 0 | 0 |
| UFC1 | 1.832 | 0 | 0 | 1.787 | 0 | 0 |
| STOM | 1.799 | 0 | 0 | 1.571 | 0 | 0 |
| PIGR | 1.791 | 0 | 0 | 1.27 | 0 | 0 |
| C9orf24 | 1.761 | 0 | 0 | 0.947 | 0 | 0.546 |
| MGST3 | 1.758 | 0 | 0 | 1.227 | 0 | 0 |
| SLPI | 1.685 | 0 | 0 | −0.284 | 0.31 | 1 |
| C11orf88 | 1.672 | 0 | 0 | 1.134 | 0 | 0 |
| C1orf194 | 1.652 | 0 | 0 | 0.967 | 0 | 0.01 |
| EFHC1 | 1.629 | 0 | 0 | 1.29 | 0 | 0 |
| C20orf85 | 1.548 | 0 | 0 | 0.797 | 0 | 1 |
| PCSK1N | 1.548 | 0 | 0 | 1.854 | 0 | 0 |
| FAM229B | 1.519 | 0 | 0 | 1.167 | 0 | 0 |
| DNAAF1 | 1.513 | 0 | 0 | 0.965 | 0 | 0 |
| CAPSL | 1.483 | 0 | 0 | 1.042 | 0 | 0 |
| PSENEN | 1.46 | 0 | 0 | 1.297 | 0 | 0 |
| UBB | 1.46 | 0 | 0 | 0.486 | 0.002 | 1 |
| MPC2 | 1.414 | 0 | 0 | 1.605 | 0 | 0 |
| AKAP9 | 1.4 | 0 | 0 | 0.972 | 0 | 0 |
| CYSTM1 | 1.387 | 0 | 0 | 1.202 | 0 | 0 |
| C9orf135 | 1.378 | 0 | 0 | 1.097 | 0 | 0 |
| ROPN1L | 1.362 | 0 | 0 | 0.998 | 0 | 0 |
| KIF9 | 1.356 | 0 | 0 | 1.107 | 0 | 0 |
| TAGLN2 | 1.336 | 0 | 0 | 2.071 | 0 | 0 |
| IK | 1.332 | 0 | 0 | 1.107 | 0 | 0 |
| CALM1 | 1.319 | 0 | 0 | 0.836 | 0 | 0.073 |
| RIIAD1 | 1.315 | 0 | 0 | 1.066 | 0 | 0 |
| APOD | 1.31 | 0 | 0 | 1.369 | 0 | 0 |
| MAP1B | 1.303 | 0 | 0 | 1.418 | 0 | 0 |
| LHX9 | 1.264 | 0 | 0 | 1.282 | 0 | 0 |
| DYNLRB2 | 1.25 | 0 | 0 | 0.786 | 0 | 0.001 |
| LRRIQ1 | 1.237 | 0 | 0 | 0.755 | 0 | 0.012 |
| TSPAN1 | 1.237 | 0 | 0 | 0.949 | 0 | 0.01 |
| CCDC170 | 1.222 | 0 | 0 | 0.87 | 0 | 0 |
| S100A11 | 1.221 | 0 | 0 | 2.008 | 0 | 0 |
| DYNLL1 | 1.21 | 0 | 0 | 1 | 0 | 0.014 |
| TUBB4B | 1.195 | 0 | 0 | 0.837 | 0 | 0.095 |
| C21orf59 | 1.17 | 0 | 0 | 1.032 | 0 | 0 |
| C12orf75 | 1.164 | 0 | 0 | 0.766 | 0 | 0.002 |
| CCDC74A | 1.159 | 0 | 0 | 0.917 | 0 | 0 |
| GDF15 | 1.157 | 0 | 0 | 1.802 | 0 | 0 |
| TUBA1A | 1.156 | 0 | 0 | 0.65 | 0 | 1 |
| FAM81B | 1.155 | 0 | 0 | 0.96 | 0 | 0 |
| CCDC78 | 1.148 | 0 | 0 | 0.735 | 0 | 0 |
| FXYD3 | 1.141 | 0 | 0 | 0.407 | 0.024 | 1 |
| CFAP36 | 1.129 | 0 | 0 | 1.044 | 0 | 0 |
| WDR54 | 1.124 | 0 | 0 | 0.805 | 0 | 0 |
| DNALI1 | 1.122 | 0 | 0 | 0.968 | 0 | 0 |
| HSP90AA1 | 1.104 | 0 | 0 | 1.018 | 0 | 0.008 |
| RSPH9 | 1.081 | 0 | 0 | 0.762 | 0 | 0 |
| CFAP45 | 1.08 | 0 | 0 | 0.884 | 0 | 0 |
| DNAH5 | 1.039 | 0 | 0 | 0.842 | 0 | 0 |
| SARAF | 1.039 | 0 | 0 | 0.693 | 0 | 0.096 |
| ANXA4 | 1.037 | 0 | 0 | 0.859 | 0 | 0 |
| SNTN | 1.033 | 0 | 0 | 0.555 | 0 | 1 |
| NUDC | 1.027 | 0 | 0 | 0.955 | 0 | 0 |
| C21orf58 | 1.02 | 0 | 0 | 0.787 | 0 | 0 |
| PPIL6 | 1.015 | 0 | 0 | 0.823 | 0 | 0 |
| RP11-295M3.4 | 1.015 | 0 | 0 | 0.693 | 0 | 0 |
| IFT22 | 1.012 | 0 | 0 | 0.953 | 0 | 0 |
| CRNDE | 1.01 | 0 | 0 | 0.523 | 0 | 1 |
| RRAD | 1.009 | 0 | 0 | 0.847 | 0 | 0 |
| CD46 | 1.006 | 0 | 0 | 1.107 | 0 | 0 |
| CRYM | 1.002 | 0 | 0 | 1.02 | 0 | 0 |
| CTSS | 1.002 | 0 | 0 | 0.816 | 0 | 0 |
| SPA17 | 1.002 | 0 | 0 | 0.767 | 0 | 0 |

1-6. Survival Analysis

To evaluate the prognostic effects of gene sets derived from specific cell states, RNA-sequencing and clinical data from patients' adenocarcinoma (LUAD) and squamous cell carcinoma (LUSC) samples were obtained from the Cancer Genome Atlas (TCGA). The RNA-seq data (Level 3) included 494 LUAD and 490 LUSC (updated in 2017) tumors, and the expression of each gene was represented as log 2(TPM+1) scale. Patients were acknowledged as survival if the time of death after diagnosis was longer than 10 years for a more refined analysis of survival rate. For each target gene, tumor samples were classified into two classes according to the 25th and 75th percentiles formulas. Survival curves were fitted using the Kaplan-Meier formula in the R package ‘survival’.

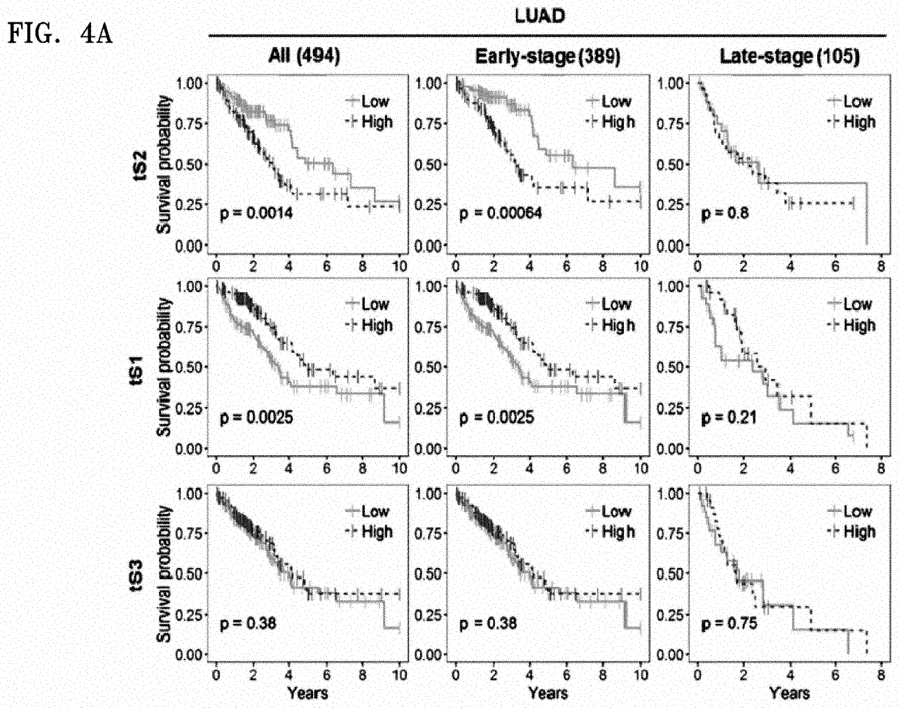

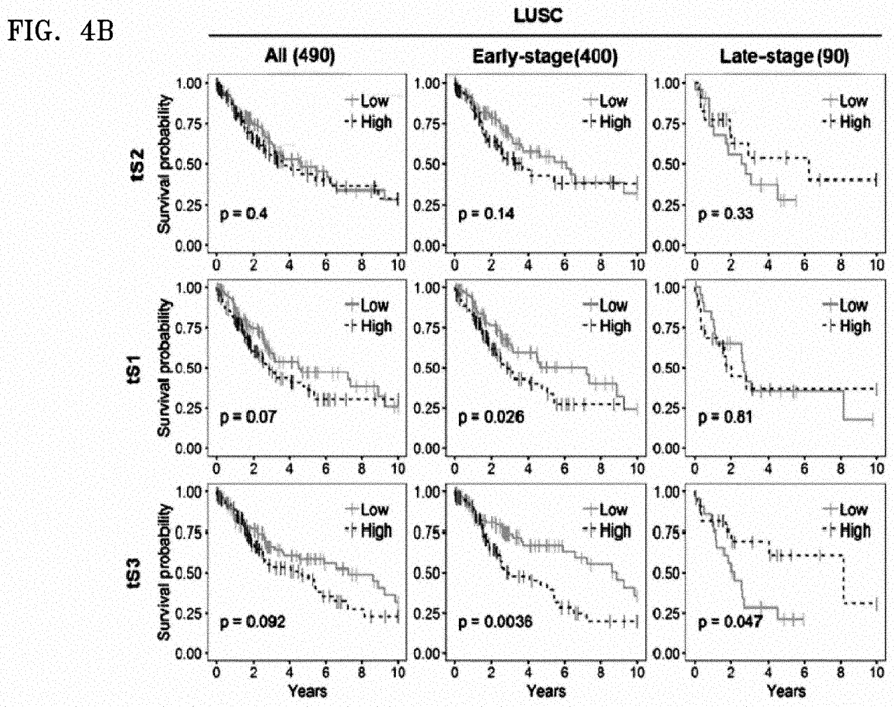

FIG. 4 are graphs showing the prognostic association of molecular subtype-specific marker genes in tumor cells. In particular, the graph shows the Kaplan-Meier survival curve for the average expression of molecular subtype-specific marker genes, and the tumor samples were annotated as ‘high’ and ‘low’ (25^{th} and 75^{th} percentiles, respectively) groups for the expression signal of each gene. P-value was determined by a log-rank test.

FIG. 4A show graphs of survival curves of lung adenocarcinoma patients. As shown in FIG. 4A, through the analysis of the independent LUAD cohort provided by TCGA, it was confirmed that overall survival rates of the patients who had high expression of the signature gene specific to the tumor cell state 2 significantly degraded (p<0.01) as compared with those of the patients who had low expression. FIG. 4B show graphs of survival curves of lung squamous cell carcinoma patients. As shown in FIG. 4B, it was confirmed that there was no difference between the signature genes specific to the tumor cell state 2 in the survival rates of the lung squamous cell carcinoma (LUSC) cohort in TCGA. Therefore, the molecular subtype analysis based on single cell trajectory analysis is applicable to predict adverse prognosis of LUAD.

1-7. Verification by Immunohistochemical Staining

To confirm whether the expression of the signature genes specific to the tumor cell state 2 in the lung adenocarcinoma (LUAD) patients increased at protein levels, immunohistochemical staining was performed on tissue samples of the lung adenocarcinoma (LUAD) patients.

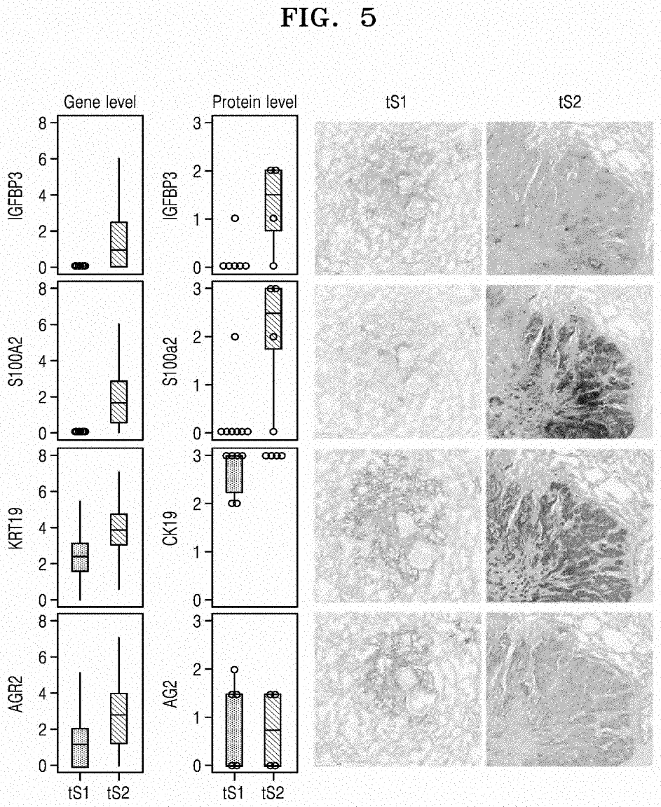

In particular, tissue samples of lung adenocarcinoma patients were fixed in 10% of formalin and embedded in paraffin. The tissue samples each corresponds to tumor cell state 1-enriched tLung (n=7) or tumor cell state 2-enriched tLung (n=4). Thereafter, 4-μm-thick sections were prepared, and proteins of IGFBP3, S100a2, CK19, and AG2 were detected using the following antibodies and dilutions: anti-IGFBP3 (mouse, 1:100, NBP2-12364, Novus Biologicals, Centennial, Colo., USA), anti-CK19 (rabbit, 1:500, NB100-687, Novus Biologicals), anti-AG2 (rabbit, 1:200, NBP2-27393, Novus Biologicals), and anti-S100a2 (rabbit, 1:300, ab109494, Abcam, Cambridge, UK). FIG. 5 shows the results of measuring expression levels of single cells and protein levels of the selected marker genes specific to tumor cell state 2 (tS2) epithelial subtypes with respect to tissue samples of patients with lung adenocarcinoma (LUAD).

As a result, as shown in FIG. 5, an increase in expression of the tumor specific to tumor cell state 2 was further confirmed at the protein level of the LUAD sample.

1. A biomarker panel comprising A biomarker panel comprising an agent measuring the level of at least two biomarkers selected from the group consisting of S100A4, TMSB10, KRT19, RAC1, S100A2, MDK, ISG15, KRT7, CLDN3, CDKN2A, and IFI27.

2. The biomarker panel of claim 1, further comprising at least one biomarker selected from the group consisting of AGR2, SOX4, C15orf48, CRIP2, HMGA1, TUBB, MARCKSL1, and IGFBP3.

3. The biomarker panel of claim 1, further comprising at least one biomarker selected from the group consisting of CSTB, S100A16, COL1A1, SPATS2L, HN1, SPINT2, PTGS2, ANXA2, and TAGLN2.

4. The biomarker panel of claim 1, wherein the biomarkers are obtained from single-cell transcriptome data.

5. The biomarker panel of claim 1, wherein the biomarkers are for diagnosing cancer.

6. The biomarker panel of claim 1, wherein the biomarkers positively regulate cell migration, apoptosis, or negatively regulate cell proliferation.

7. The biomarker panel of claim 1, wherein the agent measuring the level of the biomarkers is a primer pair, a probe, or an antisense nucleotide.

8. The biomarker panel of claim 1, wherein the agent measuring the level of the biomarkers is an antibody.

9. A method of predicting prognosis of cancer, the method comprising:
measuring the level of at least two biomarkers selected from the group consisting of S100A4, TMSB10, KRT19, RAC1, S100A2, MDK, ISG15, KRT7, CLDN3, CDKN2A, and IFI27 from a sample isolated from an individual; and
comparing the level of the biomarkers with a corresponding result of the corresponding markers in a control sample.

10. The method of claim 9, further comprising measuring the level of at least one biomarker selected from the group consisting of AGR2, SOX4, C15orf48, CRIP2, HMGA1, TUBB, MARCKSL1, and IGFBP3.

11. The method of claim 9, further comprising measuring the level of at least one biomarker selected from the group consisting of CSTB, S100A16, COL1A1, SPATS2L, HN1, SPINT2, PTGS2, ANXA2, and TAGLN2.

12. The method of claim 9, further comprising determining the prognosis as poor when the biomarkers are overexpressed as compared with the control sample.

13. The method of claim 9, wherein the cancer is lung cancer.

14. A method of determining a molecular subtype of cancer, the method comprising:
obtaining single-cell transcriptome data from a sample isolated from an individual; and
extracting a subset of genes from the data.

15. The method of claim 13, further comprising selecting a signature gene from the extracted subset of genes.

16. The method of claim 13, wherein the cancer is lung cancer.

17. Use of an agent for manufacturing a biomarker panel for predicting prognosis of cancer, wherein the agent measures the level of at least two biomarkers selected from the group consisting of S100A4, TMSB10, KRT19, RAC1, S100A2, MDK, ISG15, KRT7, CLDN3, CDKN2A, and IFI27.
